(19) United States
(12) Patent Application Publication
(10) Pub. No.: US 2010/0112545 A1
(43) Pub. Date: May 6, 2010

(54) TRANS-1,2-DIPHENYLETHLENE DERIVATIVES AND NANOSENSORS MADE THEREFROM

(76) Inventors: Subra Muralidharan, Richland, MI (US); Chun Wang, Kalamazoo, MI (US)

Correspondence Address: FLYNN THIEL BOUTELL & TANIS, P.C., 2026 RAMBLING ROAD, KALAMAZOO, MI, 49008-1631, US

(21) Appl. No.: 12/452,505

(22) PCT Filed: Jul. 13, 2007
(86) PCT No.: PCT/US2007/016067

§ 371 Date: Jan. 5, 2010

(51) Int. Cl. C12Q 1/70; C07C 309/24; C07C 63/00; C07C 69/76; C07D 213/48; C07F 7/02; C07F 3/06; C07F 3/08; C07F 7/28; C07F 15/00; C07D 217/00; C07D 209/20; C07D 219/08; G01N 33/00; G01N 21/00; G01N 27/00; C40B 60/12

(52) U.S. Cl. 435/5; 562/42; 562/459; 560/51; 546/340; 556/400; 556/118; 556/51; 556/9; 546/2; 546/139; 548/496; 546/104; 436/104; 436/164; 436/149; 506/39; 977/774; 977/773

(57) ABSTRACT

Novel trans-1,2-diphenylethylene derivatives are synthesized which can be used to form nanoparticles-monomer-nanomolecule-receptor nanosensors. These trans-1,2-diphenyl-ethylene derivatives are soluble in both water and organic solvents, highly fluorescent and can be synthesized in high yields. The trans-1,2-diphenylethylene derivatives are bonded to a nanoparticle, a nanomolecule bonded to the derivative and a receptor bonded to the nanomolecule to form a nanosensor that can be used to detect chemical and biological agents.

### CROSS REFERENCE TO RELATED APPLICATION

This application is a 35 USC 371 nationalization of PCT Application No. PCT/US2007/016067, filed Jul. 13, 2007, which international application published in English.

### BACKGROUND OF THE INVENTION

1. Field of the Invention

The present invention pertains to trans-1,2-diphenyl-ethylene derivatives and nanosensors capable of detecting chemical and biological agents and sensors formed from the derivatives.

2. Description of the Related Art

Trans-1,2-diphenylethylene, hereinafter referred to as stilbene, has been conventionally used in the manufacture of dyes, optical brighteners, as a phosphor and a scintillator and as a gain medium in dye lasers.

Recently, stilbene-based compounds have been investigated for their properties in the field of molecular electronics and photonics. Viau et al, Tetrahedron Letters 45 (2004), pgs. 125-128, discusses the synthesis, optical and thermal properties of bipyridine chromophores featuring oligophenylenevinylene conjugated groups.

Dudek et al, J. Am. Chem. Soc. 2001, 123, pgs. 8033-8038, discloses the preparation of ferrocene-terminated oligophenylenevinylene methyl thiols which can possibly have a utility in the design of biosensors and molecular devices.

Tew et al, J. Am. Chem. Soc. 1999, 121, pgs. 9852-9866, discloses the synthesis of triblock rodcoil molecules containing conformationally rigid and flexible sequences and luminescent chromophores based on phenylene vinylene and the interest in these compounds due to the electronic and optical properties.

Vo-Dinh, The 1^{st} International Symposium on Micro & Nano Technology, 14-17 Mar., 2004, Honolulu, Hi., USA, discusses the development of biosensors, nanosensors and biochips for chemical, biological and medical analysis and discloses that surface-enhanced fluorescence can be used as an indicator.

### BRIEF SUMMARY OF THE INVENTION

It is an object of the present invention to provide novel stilbene derivatives which are highly fluorescent and can be used in the formation of nanosensors.

It is a further object of the present invention to provide a nanosensor for detecting chemical and biological agents which is formed from a nanoparticles, the novel stilbene derivatives, a nanomolecule and a receptor for the chemical or biological agent.

### BRIEF DESCRIPTION OF DRAWINGS

FIG. 1 is a drawing illustrating the construction of the nanosensor of the present invention;

FIG. 2 is a drawing illustrating the operation of the fluorescent sensor indicating means of an embodiment of the present invention;

FIG. 3 is a drawing illustrating a chip sensor according to the present invention;

FIG. 4 is a drawing illustrating a fiber sensor according to the present invention;

FIG. 5 is a graph showing the response of a sensor to the present invention based on the concentration of dichloropropene; and

FIG. 6 shows the response of another embodiment of a sensor according to the present invention to dichloropropene.

FIGS. 7a and 7b show the response of another embodiment of a sensor according to the present invention to dichloropropene.

FIG. 8 shows the response of another embodiment of a sensor according to the present invention to dichloropropene.

### DETAILED DESCRIPTION OF THE INVENTION

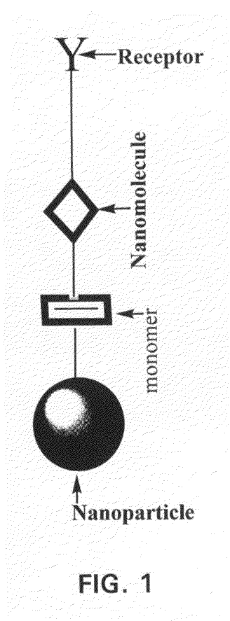

The present invention is based on the discovery that a novel family of fluorescent stilbene monomers can be used to form a nanosensor capable of releasing a fluorescent signal upon the detection of a chemical or biological agent. As shown in FIG. 1, the nanosensor of the present invention comprises a nanoparticle, the novel stilbene monomer of the present invention bonded thereto, a nanomolecule bonded to the stilbene monomer and a receptor bonded to the nanomolecule.

As the nanoparticle, particles having a size range of about 5 to 100 nanometers can be used. As the material of the nanoparticles, any material which can serve as a substrate to which the inventive stilbene monomer can be attached to can be used. Preferable materials are silica, semiconductor quantum dots, zinc sulfide and cadmium sulfide doped with various metal ions, titanium dioxide, silica-gold, gold-silica and ferromagnetic iron oxide.

The surface of the nanoparticles are functionalized so that the inventive stilbene monomer can be attached thereto. The functionalizing agent is not critical as long as it is capable of forming a bond between the nanoparticles and the stilbene monomer. A preferred functionalizing agent is 3-aminopropyltrimethoxysilane. The nanoparticles can be derivatized with the functionalizing agent in order to introduce the functional groups thereon or, as discussed above, can be obtained having the functional agents already introduced thereon.

The nanosensor as indicated in FIG. 1 is synthesized bottom-up. The nanoparticles are derivatized with a suitable linker such as triethoxyaminopropyl silane in the case of silica nanoparticles to which the stilbene monomer of choice is attached. Alternatively, with nanoparticles such as quantum dots and nanoparticles having magnetic properties, the stilbene can be directly attached without a linking molecule. The metal complex with a suitable receptor can then be ion-paired with the stilbene or the receptor directly attached to the stilbene to generate Nanoparticle-fluorescent Monomer-Nanomolecule-Receptor (NMNR) and Nanoparticle-fluorescent Monomer-Receptor (NMR) sensors respectively where signal amplification upon the interaction of the receptor with the target occurs by signal transduction. These sensors can be formed as an array on a quartz chip by dispersing them in a solvent like methanol and depositing them either by spin or dip coating.

The novel stilbene monomers of the present invention are shown by the below formulas (1)-(6) and soluble in either water or an organic solvent. These monomers can be synthesized as shown in the reaction schemes below.

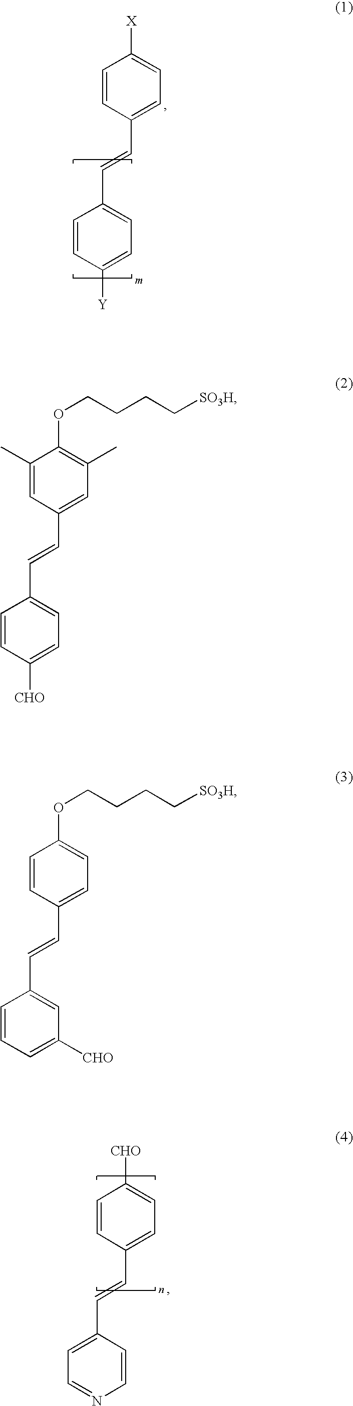

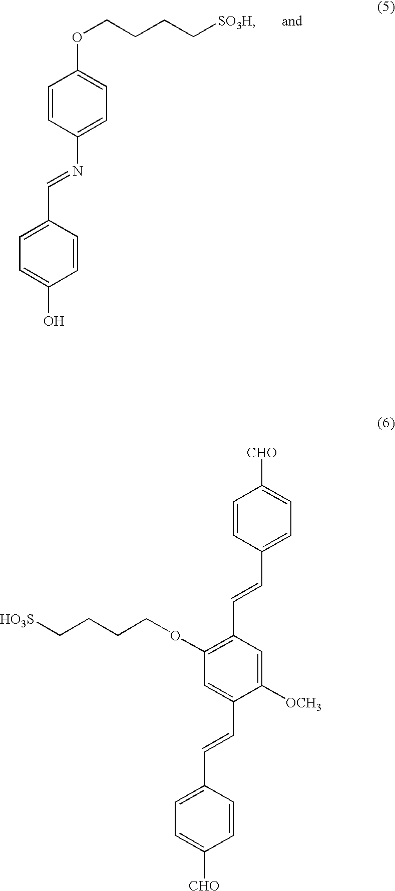

### SYNTHESIS EXAMPLE 1

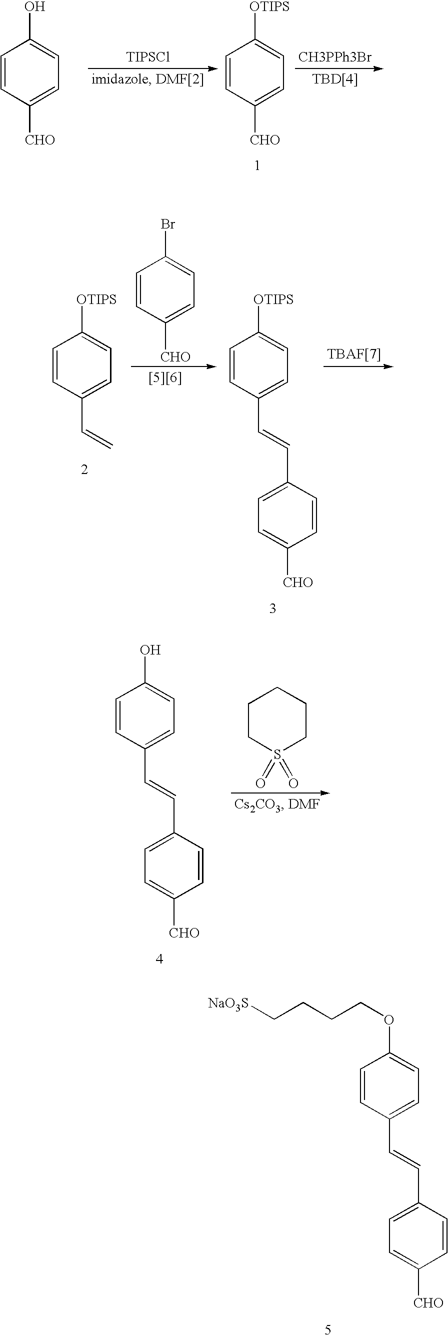

### Synthesis of 4-(triisopropylsilyloxyl)benzaldehyde 1

To a stirred solution of 1.22 g (0.01 mol) of 4-hydroxylbenzaldehyde in anhydrous DMF (25 ml) at room temperature, 0.817 g (0.012 mol) of imidazole, and 2.33 ml (0.011 mol) of tri-isopropyl silyl chloride were added. The mixture was stirred for 12 hours at room temperature. Extraction with 50 ml of ether was followed by washing with 100 ml of water three times. The organic layer was dried with magnesium sulfate. The ether was removed under vacuum to obtain a colorless oil 1 of 2.64 g (yield=95%). ¹H NMR (400 MHz, CDCl₃): 9.86 (s, 1H), 7.78 (d, 2H), 6.96 (d, 2H), 1.25 (m, 3H), 1.08 (d, 18H).

### Synthesis of 4-(triisopropylsilyloxyl)styrene 2

To a solution of 1.97 g (0.0055 mol) of methyl triphenyl phosphonium bromide in 25 ml of anhydrous THF, 0.84 g (0.006 mol) of 1, 3, 4, 6,7,8-Hexahydro-2H-pyrimido(1,2-a)-pyrimidine was added. After stirring for 15 minutes, 1.39 g (0.005 mol) of 4-(triisopropylsilyloxyl)benzaldehyde (1) was added. The mixture was stirred for 12 hours under reflux. It was extracted with 50 ml of chloroform, followed by washing 100 ml water three times. The organic layer was dried over MgSO₄, solvent removed under vacuum, the residual brown oil was purified by column chromatography on silica gel (chloroform) to yield 1.01 g (yield=73%) of pale oil 2. ¹H NMR (400 MHz, CDCl₃): 7.26 (d, 2H), 6.83 (d, 2H), 6.65 (m, 1H), 5.61 (d, 1H), 5.11 (d, 1H), 1.23 (m, 3H), 1.08 (d, 18H).

### Synthesis of Stilbene Compound 3

To a solution of 0.552 g (0.002 mol) of silyl ether styrene (2) in 20 ml of anhydrous DMF, 0.37 g (0.002 mol) of 4-bromobenzaldehyde, 0.009 g (0.04 mmol, 2% of 2) of palladium acetate, 0.0244 g (0.08 mmol, 4% of 2) of tri-o-tolylphosphine, 0.42 ml (0.003 mol, 1.5 equivalent) of triethylamine were added in order under stirring. The mixture was heated for 24 hours at 110° C., and filtered through celite 545 packed funnel at room temperature. It was extracted with 40 ml of chloroform, followed by washing with 100 ml of water three times. The organic layer was dried over MgSO₄. After removing the solvent under vacuum, the stilbene compound 3 was isolated by column chromatography on silica gel (chloroform) with a yield of 40% (0.3 g). ¹H NMR (400 MHz, CDCl₃): 10.02 (s, 1H), 7.84 (d, 2H), 7.61 (d, 2H), 7.42 (d, 2H), 7.20 (d, 1H), 7.00 (d, 1H), 6.89 (d, 2H), 1.25 (m, 3H), 1.10 (d, 18H).

### Deprotection of Silyl Ether Stilbene 4

To a solution of 0.38 g (0.001 mol) of silyl ether styrene 3 in anhydrous THF, 1 ml (0.001 mol, 1M in THF) of tetrabutylammonium fluoride was added drop wise under nitrogen. After stirring for 5 minutes, the reaction was quenched with acetic acid/ether. THF was removed under vacuum. The deprotected hydroxyl stilbene was precipitated out in ether and filtered to yield 0.2 g (yield=86%) of hydroxyl stilbene 4. ¹H NMR (400 MHz, DMSO-d6): 10.05 (s, 1H), 9.75 (s, 1H), 7.97 (d, 2H), 7.76 (d, 2H), 7.5 (d, 2H), 7.39 (d, 2H), 7.13 (d, 2H), 6.80 (d, 2H).

### Preparation of Final Product 5

To a solution of 0.2 g (0.9 mmol) of hydroxyl stilbene 4 in anhydrous DMF, 0.18 g (0.5 mol, 1.2 equivalent) of cesium carbonate was added. After stirring for 30 minutes, 0.11 ml (1.1 mmol, 1.2 equivalent) of 4-butanesultone was added under nitrogen. After stirring for 12 hours, the reaction was quenched with drops of HCl/ether. The final product was precipitated in ether and collected by filtration with a yield of 83% (0.28 g). ¹H NMR (400 MHz, DMSO-d6): 9.99 (s, 1H), 7.88 (d, 2H), 7.79 (d, 2H), 7.59 (d, 2H), 7.43 (d, 1H), 7.21 (d, 1H), 6.97 (d, 2H), 3.98 (t, 2H), 2.48 (t, 2H), 1.79 (m, 4H).

The overall yield was 20% through five steps.

### SYNTHESIS EXAMPLE 2

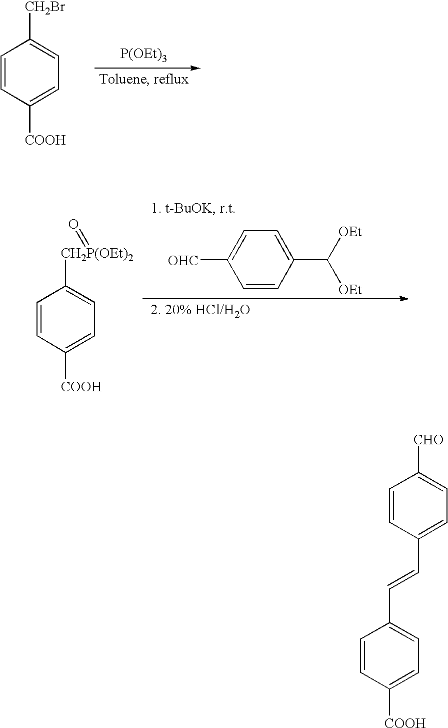

### SYNTHESIS EXAMPLE 3

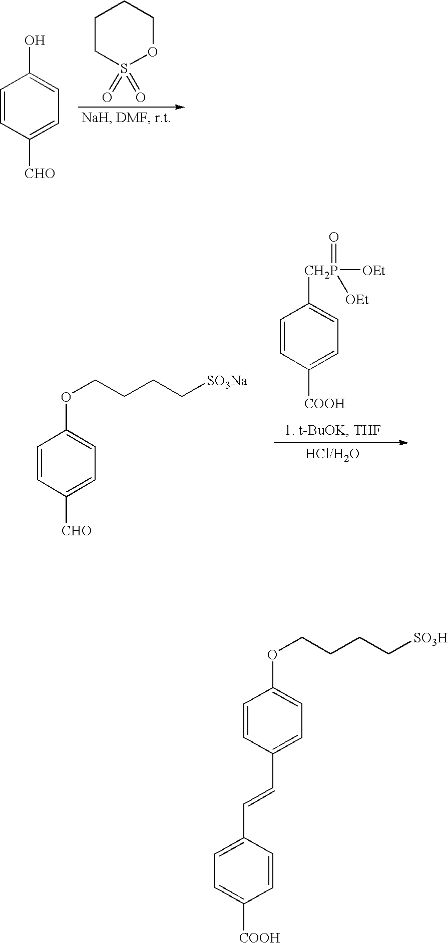

### SYNTHESIS EXAMPLE 4

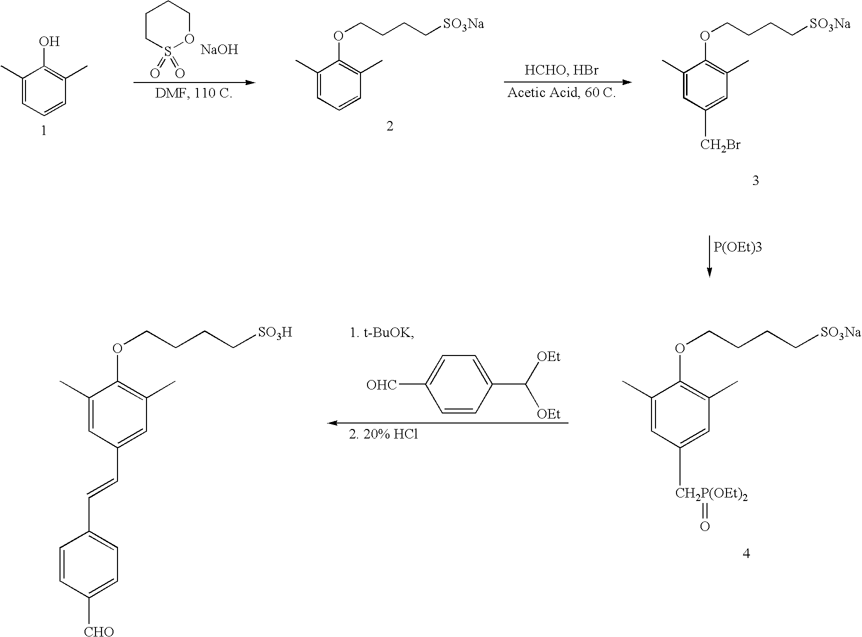

### SYNTHESIS EXAMPLE 5

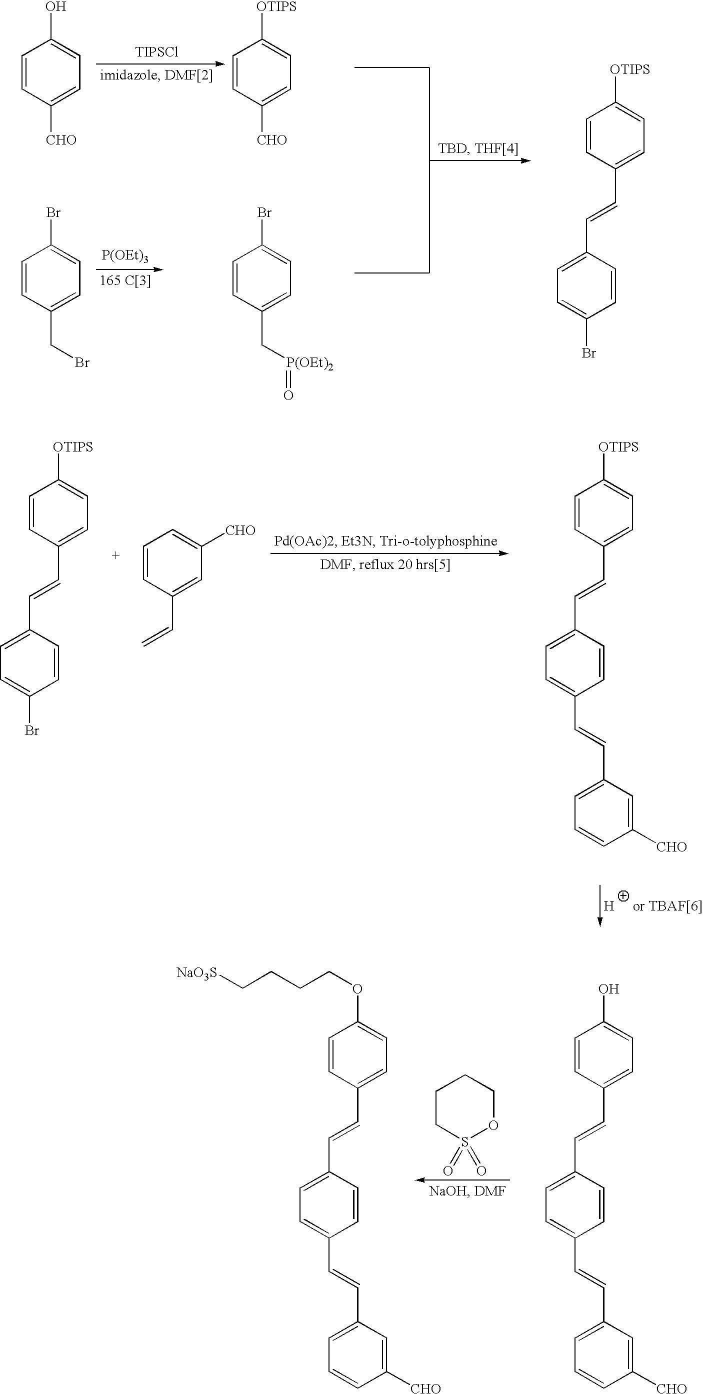

### SYNTHESIS EXAMPLE 6

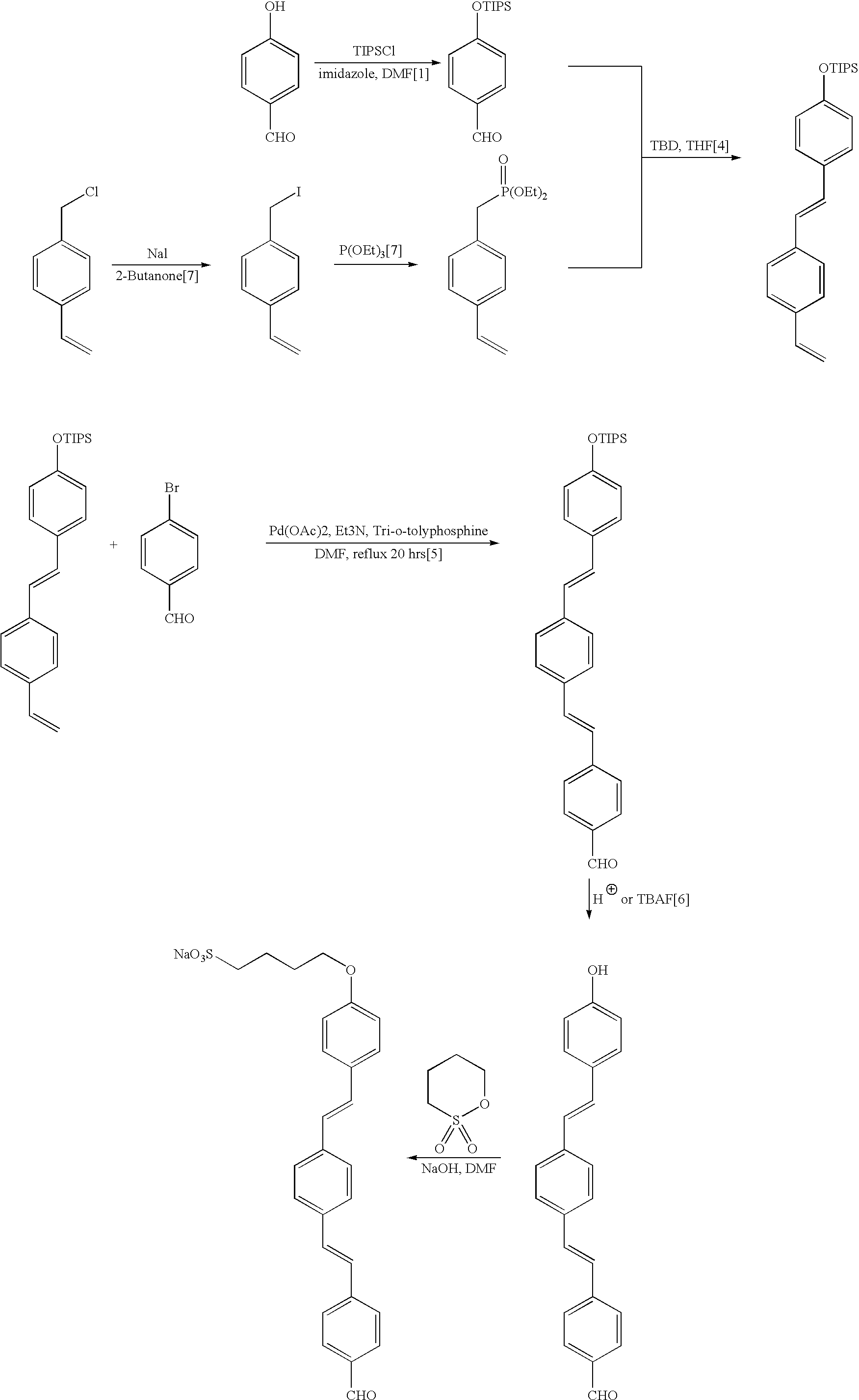

### SYNTHESIS EXAMPLE 7

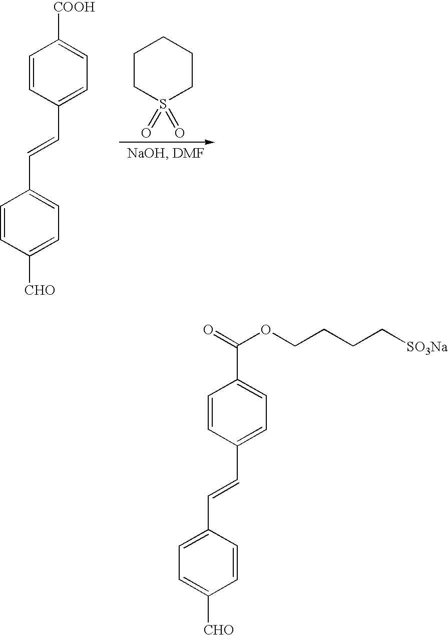

### SYNTHESIS EXAMPLE 8

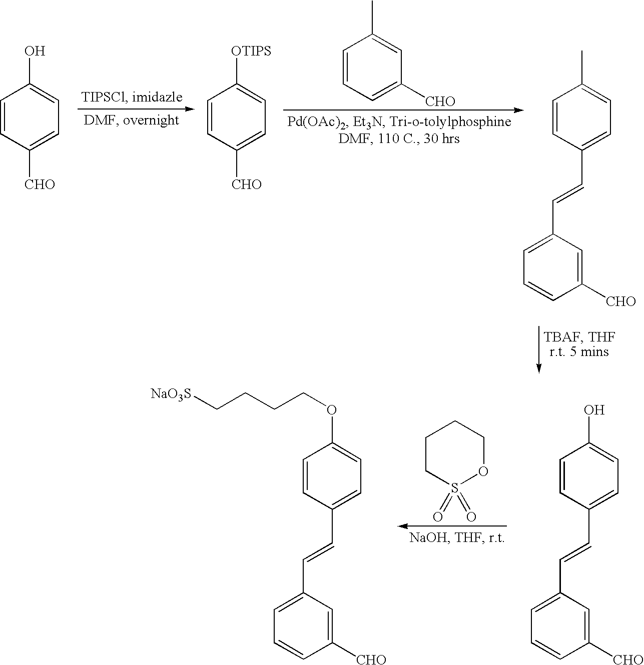

### SYNTHESIS EXAMPLE 9

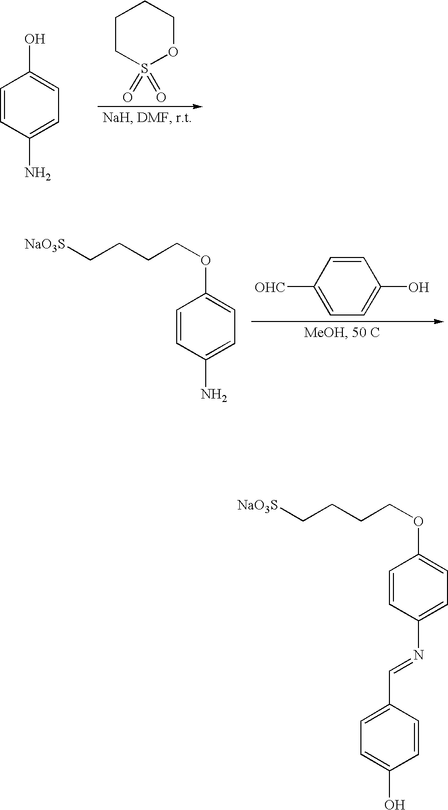

### SYNTHESIS OF EXAMPLE 10

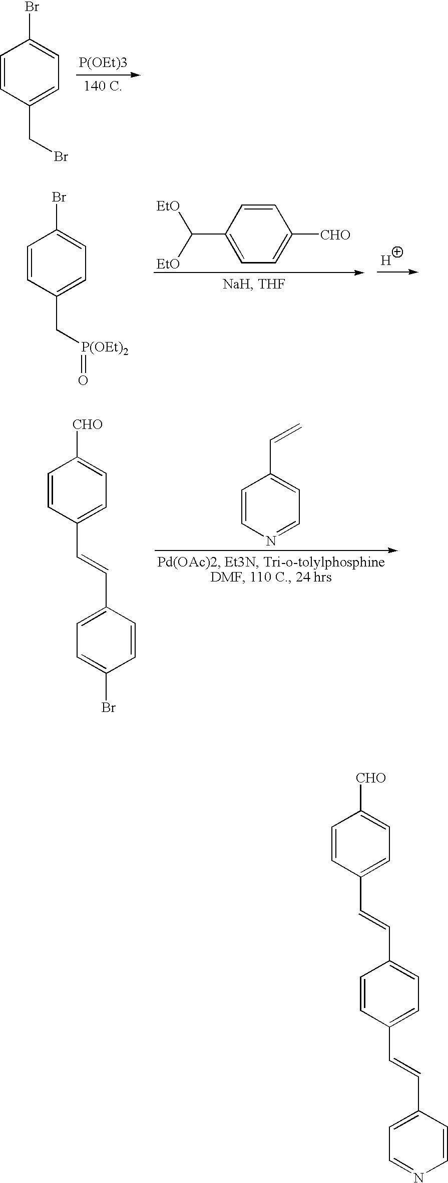

As the nanomolecule which joins the novel stilbene derivative of the present invention with the receptor, any suitable compound can be used. Bipyridyl compounds such as a ruthenium bipyridyl compound or a zinc bipyridyl compound are particularly preferred.

The receptor to be attached to the nanomolecule is selected depending on the target, namely a chemical or a biological agent, and could be readily determined by one of ordinary skill in the art. These receptors include isoquinolene, tryptophan methyl ester, 9-amino acridine, fluoresceinamine, 2-amino-5-hexafluoroisopropanol-cyclohexa-1,4 diene and bis(2,2′-amino-3,3′-hydroxy-1-5,5′-hexafluoro-isopropyl)-cyclohexa-1,4 diene.

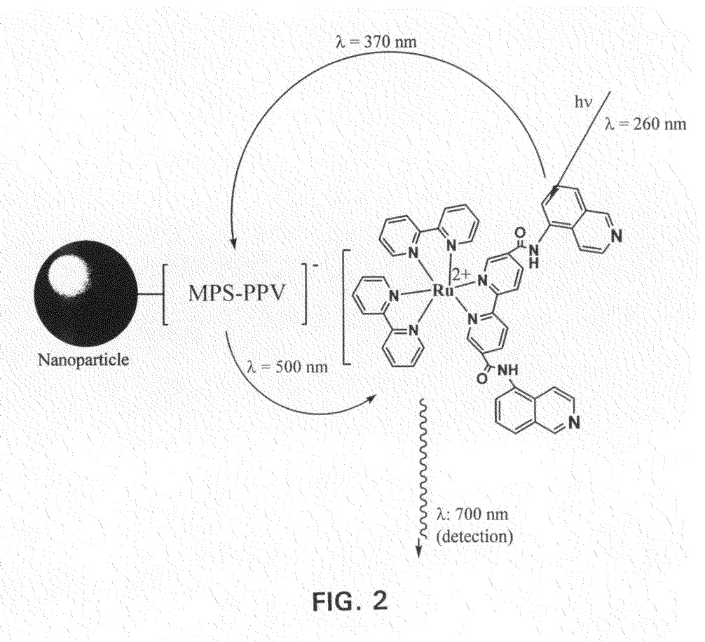

FIG. 2 illustrates the fluorescence sensing mechanism of the present invention in which a ruthenium bipyridyl compound is used as a nanomolecule and isoquinoline is used as the receptor of the target gas or chemical agent in a solution or vapor phase.

The stilbene derivative sensors of the present invention can be embedded into swabs which are then used to collect fluids for direct analysis. Membranes can be embedded with the sensors to detect viruses, such as influenza and pox viruses, from the breath of a subject. The output signals of the sensors of the present invention could be optical, i.e., absorption and emission, electroluminescent, magnetic, and acoustic (photoacoustic and magnetoacoustic), either generated independently or simultaneously.

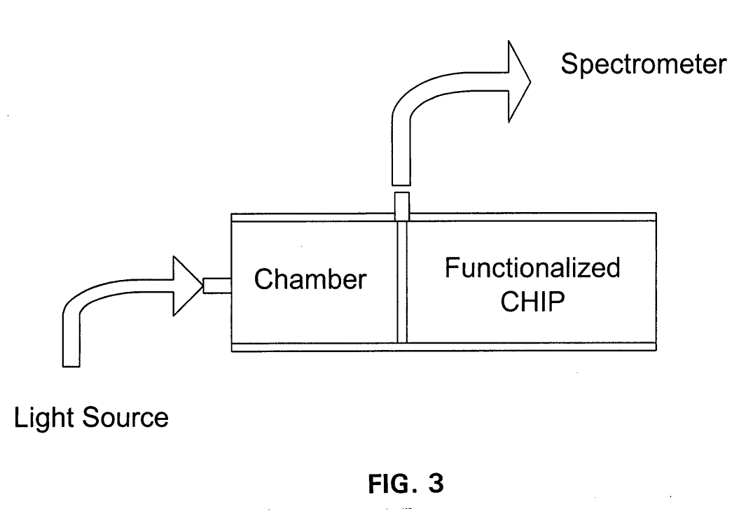

FIG. 3 illustrates the operation of a chip sensor in which an excitation light source is used to cause fluorescence of a functionalized chip containing the nanosensor of the present invention in the presence of a target agent.

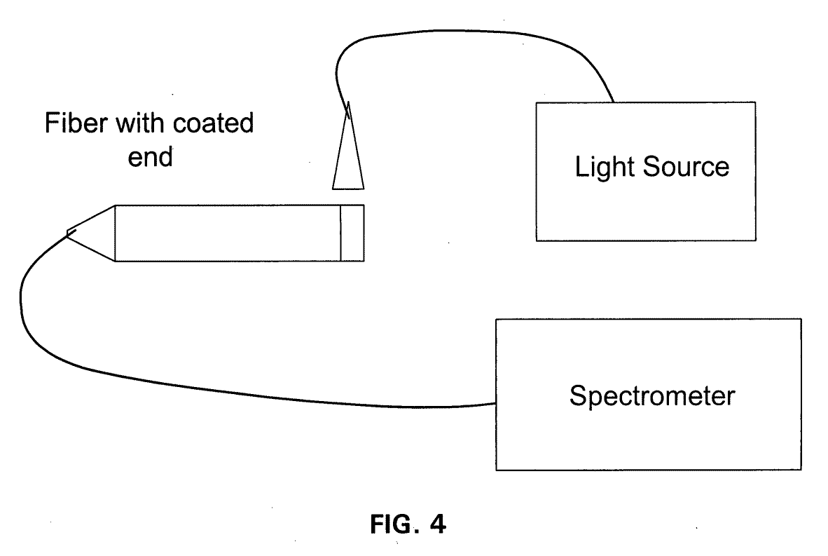

FIG. 4 illustrates a fiber sensor using the nanosensors of the present invention in which a fiber is provided with an end coated with the nanosensors of the present invention and the fluorescence of the nanosensors measured by a spectrometer in the presence of an excitation light source and the target agent.

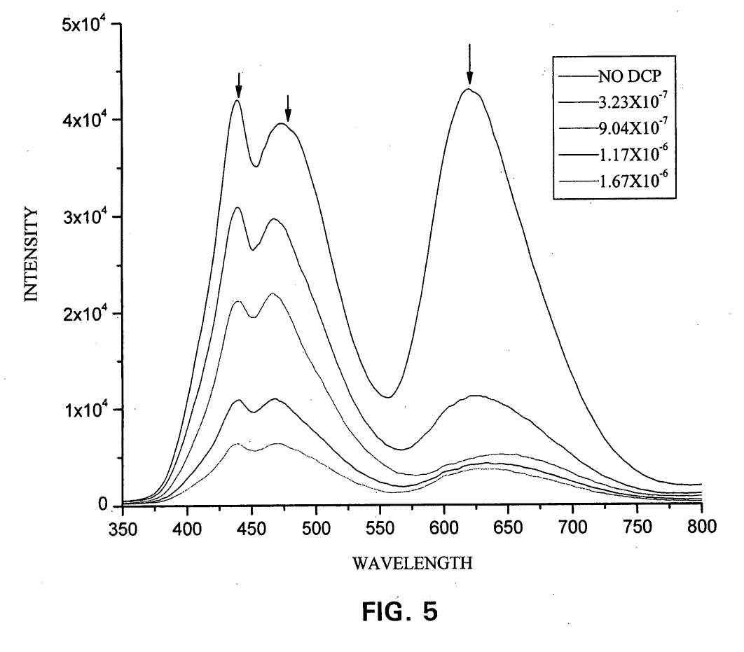

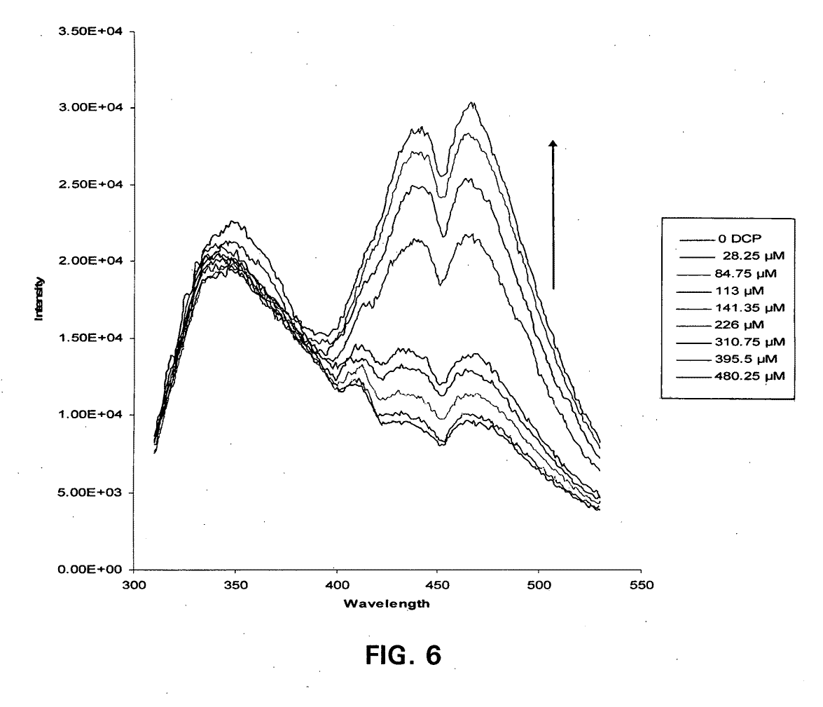

FIGS. 5 and 6 illustrate the fluorescent response of sensors according to the present invention in the presence of different concentrations of diethoxychlorophosphate (DCP), which illustrates the sensitivity of the nanosensors of the present invention to minute concentrations of the chemical agent.

Specifically speaking, FIG. 5 is a graph showing the response of a chip sensor of the present invention based on the concentration of DCP. The chip sensor is formed by a nanosensor array provided on a quartz plate. The sensor is formed from silica nanoparticles, an inventive stilbene monomer, a ruthenium complex and an isoquinoline receptor. The DCP is detected by a decrease in fluorescence and the sensor is of the “switch-off” type. The association constant is 8.9×10⁵1M⁻¹.

FIG. 6 is a graph showing the response of a sensor of the present invention based on the concentration of DCP and is of the “switch-on” type in which the DCP is detected by an increase in fluorescence of the sensor.

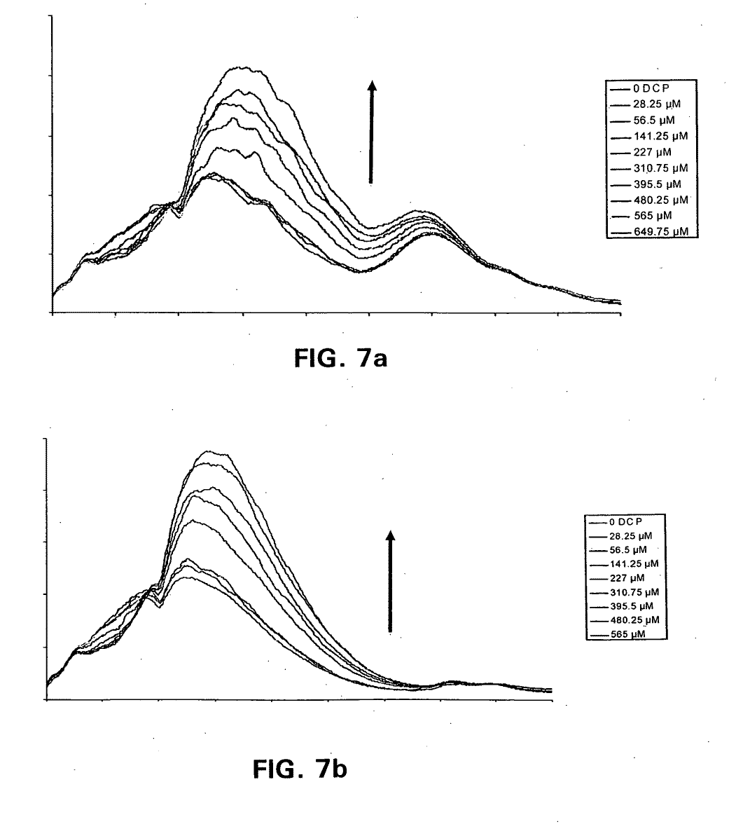

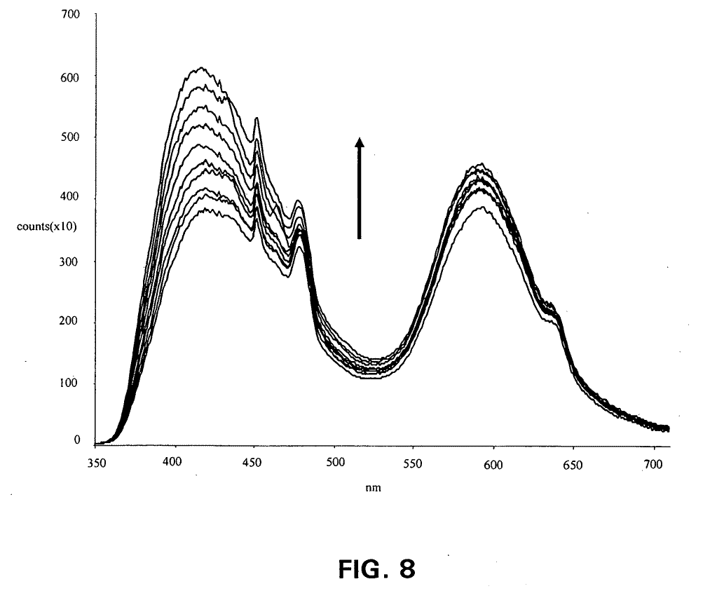

FIGS. 7a and 7b are graphs showing the response of sensors of the present invention based on the concentration of DCP and are of the “switch-on” type in which the DCP is detected by an increase in fluorescence of the sensor. The sensors of FIGS. 8a and 8b have the construction as shown below and consist of a silica nanoparticle, a novel stilbene monomer, a ruthenium complex and a tryptophan receptor for the sensing of DCP. The ruthenium complex of FIG. 8(a) is a 4,4′ complex and the association constant is K=4.66×10²M⁻¹. The ruthenium complex of FIG. 8(b) is a 5,5′ complex and the association constant is 1.631×10³M⁻¹. The two sensors differ in that the tryptophan receptor is present at two different positions of the bipyridyl ring of the ruthenium complex.

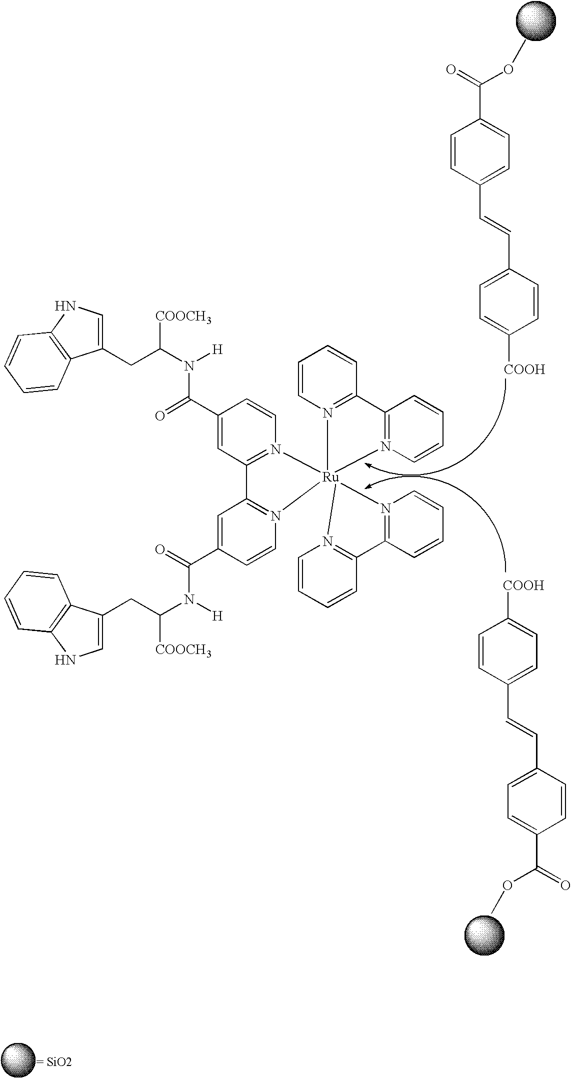

FIG. 8 is a graph showing the response of a sensor according to the present invention based on the concentration of DCP. The sensor has the construction shown below and is a “switch-on” type sensor which exhibits an increase in fluorescence upon the detection of DCP. The sensor is made of ZnS:Mn/ZnS core/shell quantum dots as nanoparticles, a novel stilbene monomer and an isoquinoline receptor and has an association constant of K=2.2×10³M⁻¹.

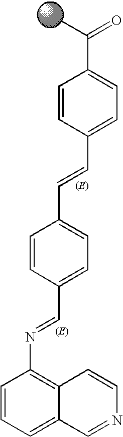

Although the novel stilbene derivative of the present invention has been extensively described above for use in a nanosensor, the utility thereof is not limited to nanosensors as the inventive stilbene derivatives also have utilities as organic light emitting diodes, electroluminescence, biomarkers and organized molecular self-assemblies for nanomaterial synthesis. The novel stilbene derivatives of the present invention are soluble in both water and organic solvents and their synthesis can be controlled to obtain the target molecules in high yield and readily introduce various functional groups therein to modify their properties.

What is claimed is:

1. A trans-1,2-diphenylethylene derivative of the formula (1), (2), (3), (4), (5) or (6), 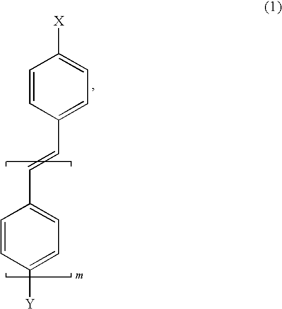
wherein X is CHO, O(CH₂)₄SO₃H or CO₂(CH₂)₄SO₃Na, Y is CO₂H, CHO or OH, and m is 1 or 2, with the proviso that when X is CHO and Y is CO₂H, m is 1, 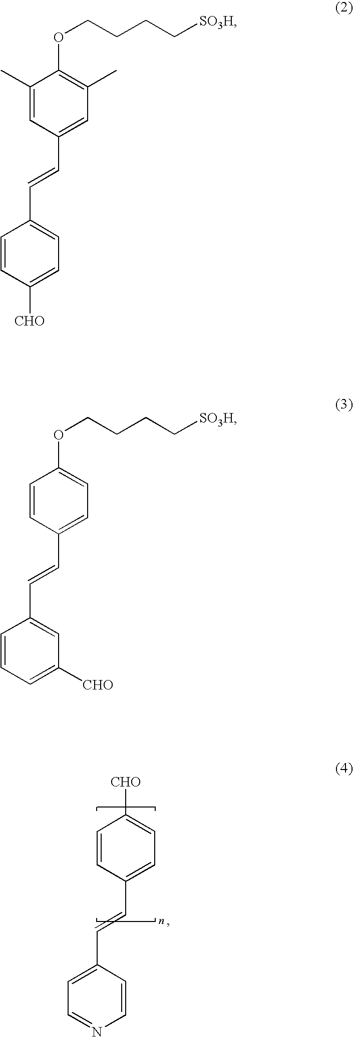
wherein n is 1 or 2,

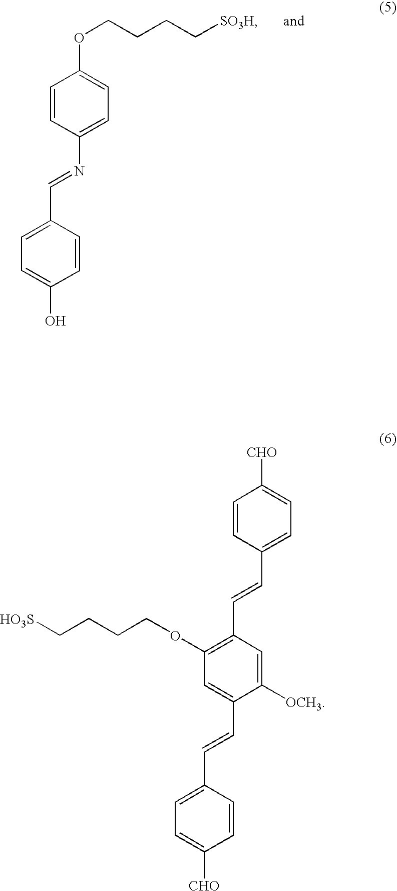

2. The stilbene derivative of claim 1, wherein said derivative is of formula (1).

3. The stilbene derivative of claim 1, wherein said derivative is of formula (2).

4. The stilbene derivative of claim 1, wherein said derivative is of formula (3).

5. The stilbene derivative of claim 1, wherein said derivative is of formula (4).

6. The stilbene derivative of claim 1, wherein said derivative is of formula (5).

7. A nanosensor for detecting the presence of chemical and biological agents comprising the stilbene derivative of claim 1.

8. The nanosensor of claim 7, comprising a nanoparticle, the water-soluble stilbene derivative bonded to the nanoparticle, a nanomolecule bonded to the stilbene derivative and a receptor bonded to the nanomolecule.

9. The nanosensor of claim 8, wherein said nanoparticle is at least one member selected from the group consisting of silica, zinc sulfide, cadmium sulfide, titanium dioxide and silica-gold.

10. The nanosensor of claim 8, wherein the nanomolecule is at least one of a ruthenium (II) bipyridinyl complex and a zinc (II) bipyridinyl complex.

11. The nanosensor of claim 8, wherein the receptor is at least one member selected from the group consisting of isoquinoline, tryptophan methyl ester, 9-amino acridine, fluoresceinamine, 2-amino-5-hexafluoroisopropanol-cyclohexa-1,4 diene and bis(2,2′-amino-3,3′-hydroxy-1-5,5′-hexafluoroisopropyl)-cyclohexa-1,4 diene.

12. The nanosensor of claim 8, wherein said nanoparticle is a quantum dot.

13. In a method of detecting the presence of chemical or a biological agent using a nanosensor, the improvement comprising said nanosensors comprising the trans-1,2-diphenyl-ethylene derivative of claim 1.

14. The method of claim 13, wherein the nanosensor is used to detect a chemical agent.

15. The method of claim 13, wherein the nanosensor is used to detect a biological agent.

16. The method of claim 15, wherein the biological agent is a pox or an influenza virus.

17. The method of claim 14, wherein the chemical agent is a diethoxychlorophosphate.

18. The method of claim 13, wherein said nanosensor emits an optical signal upon detection of the agent.

19. The method of claim 13, wherein said nanosensor emits an electroluminescent signal upon detection of the agent.

20. The method of claim 13, wherein said nanosensor emits a magnetic signal upon detection of the agent.

21. The method of claim 13, wherein said nanosensor emits an acoustic signal upon detection of the agent.

22. The method of claim 13, wherein said nanosensor is embedded in a swab.

23. The method of claim 13, wherein said nanosensor is embedded in a membrane.

24. A sensor array for detecting the presence of chemical and biological agents comprising a plurality of nanosensors according to claim 8 provided on a substrate.
